(19) United States
(12) Patent Application Publication
(10) Pub. No.: US 2026/0002011 A1
(43) Pub. Date: Jan. 1, 2026

(54) SOLVENT REMOVAL IN A PROCESS FOR RECYCLING ONE OR MORE POLYMERS, SUCH AS POLYURETHANES, CONTAINED IN A SOLID MATERIAL

(71) Applicant: BASF SE, Ludwigshafen am Rhein (DE)

(72) Inventors: Patrick SCHMATZ-ENGERT, Ludwigshafen am Rhein (DE); Jens FERBITZ, Ludwigshafen am Rhein (DE); Matthias Maximilian NIEDERMAIER, Ludwigshafen am Rhein (DE); Torsten MATTKE, Ludwigshafen am Rhein (DE); Christian DITTRICH, Ludwigshafen am Rhein (DE); Michaela STUPPY, Ludwigshafen am Rhein (DE)

(21) Appl. No.: 18/880,754

(22) PCT Filed: Jul. 4, 2023
(86) PCT No.: PCT/EP2023/068413

§ 371 Date: Jan. 2, 2025

(30) Foreign Application Priority Data
Jul. 5, 2022 (EP) 22183136.5

(51) Int. Cl. C08J 11/28; C07C 209/62; C07C 209/86; C08J 11/14; C08J 11/20

(52) U.S. Cl. CPC C08J 11/28; C07C 209/62; C07C 209/86; C08J 11/14; C08J 11/20; C08J 2475/04

(57) ABSTRACT

The present invention relates to a solvent-efficient process for recycling one or more polymers selected from the group consisting of polyurethanes, polyurethane ureas, polyisocyanurates, and a mixture of two or more thereof, said one or more polymers being contained in a solid material W.

The present invention relates to a solvent-efficient process for recycling one or more polymers selected from the group consisting of polyurethanes, polyurethane ureas, polyisocyanurates, and a mixture of two or more thereof, said one or more polymers being contained in a solid material W.

Polyurethanes belong to the class of polycondensation polymers. They are generally produced from one or more polyhydroxyl compounds and one or more di- or polyisocyanates.

In terms of sustainability, the polyurethanes should be recycled as much as possible. Generally, polyurethanes can be recycled in a variety of ways. The re-monomerization of polyurethane or polyurethane waste with the recovery of polyol and isocyanate components or corresponding precursors is one of the most interesting ways. For this purpose, the prior art proposes the cleavage of the polyurethanes by, for example, hydrolysis, glycolysis, alcoholysis and aminolysis, to form an isocyanate derivative (amine, carbamate, urea) and the polyol.

For example, WO 2008/014988 A1 relates to the redissociation of polyurethanes. In particular, a process is described for splitting polyurethanes and polyurethane ureas, in which the polymer is first reacted with gaseous or liquid secondary aliphatic or cycloaliphatic amines, the secondary urea formed, after removal, is split with hydrogen chloride to the isocyanate. Further, the polyols or polyamines also formed in the reaction are worked up and purified.

One of the biggest challenges of the prior art recycling processes is the separation of the polyols from the isocyanate derivatives, which is mostly based on a phase decay. However, this can be applied only for selected polyurethanes and it can easily fail due to possible contamination in the polyurethane waste. Thus, the prior art recycling processes are typically severely limited with respect to their applicability.

Thus, there is a need for an improved process for recycling polymers such as polyurethanes, polyurethane ureas and polyisocyanurates. Accordingly, it was an object of the present invention to provide an improved process for the recycling of polymers such as polyurethanes, polyurethane ureas and polyisocyanurates, in particular for the decomposition of said polymers into monomers in order to produce new polymers.

Surprisingly, it has been found that the process of the present invention permits to recycle a variety of polyurethanes, polyurethane ureas, polyisocyanurates from solid waste material, such as end-of-life foam, by decomposing said polymers which can then be used for different applications such as the production of new monomers. Further, the process of the present invention permits to reduce the overall solvent consumption compared to known processes for recycling polymers, which reduction also permits to reduce costs. Hence, using a solvent-efficient process for recycling polymers such as polyurethanes, polyurethane ureas and polyisocyanurates permits to reduce the CO₂ footprint.

Therefore, the present invention relates to a solvent-efficient process for recycling one or more polymers, selected from the group consisting of polyurethanes, polyurethane ureas, polyisocyanurates, and a mixture of two or more thereof, said one or more polymers being contained in a solid material W, the process comprising
(i) preparing a mixture M comprising one or more polyurea-containing compounds, and further comprising one or more polyols, comprising:
   (i.1) providing a solid material W comprising one or more polymers selected from the group consisting of polyurethanes, polyurethane ureas, polyisocyanurates, and a mixture of two or more thereof;
   (i.2) introducing the solid material W provided in (i.1) and one or more primary amines, as a first solvent, in a reactor unit R_{A}, obtaining a mixture;
   (i.3) subjecting the mixture obtained according to (i.2) in R_{A} to aminolysis reaction conditions, obtaining a mixture M comprising one or more polyurea-containing compounds, and further comprising one or more polyols and one or more primary amines;
   (i.4) removing the mixture M obtained according to (i.3) from R_{A};
(ii) passing a second solvent S and the mixture M obtained according to (i) into an evaporation unit EU, obtaining, from the evaporation unit EU, a vapor mixture M_{V} comprising S and at least a portion of the one or more primary amines, and a liquid mixture M_{L} comprising the one or more polyurea-containing compounds, and the one or more polyols;
(iii) isolating the one or more polyurea-containing compounds from M_{L} obtained according to (ii), obtaining a mixture U comprising the one or more polyurea-containing compounds and a mixture P comprising the one or more polyols;
(iv) subjecting the mixture U comprising the one or more polyurea-containing compounds obtained according to (iii) to cleavage reaction conditions in a reaction unit R_{C}, obtaining either one or more corresponding polyamines or one or more corresponding polyisocyanates;
(v) separating the one or more primary amines from M_{V} obtained according to (ii) in a purification unit PU, obtaining a vapor mixture A comprising the one or more primary amines depleted in S;
(vi) recycling at least a part of A obtained according to (v) from PU into R_{A} in (i.2) as the first solvent.

Preferably, the process further comprises, prior to (i), subjecting the solid material W to a pre-treatment, more preferably a mechanical pre-treatment, wherein the mechanical pre-treatment more preferably comprises one or more of milling, crushing, shredding and cutting, more preferably milling or shredding, of the solid material W.

Preferably, the process further comprises, prior to (i), more preferably after subjecting the solid material W to a pre-treatment as defined in the foregoing, drying the solid material W, wherein drying is more preferably conducted at a temperature in the range of from 40 to 100° C., more preferably in the range of from 50 to 85° C., and wherein more preferably drying is conducted in a gas atmosphere comprising one or more of nitrogen and oxygen, more preferably air.

Preferably, the water content of the solid material W, more preferably after drying as defined herein above, is lower than 1000 ppm-weight-%, lower than 100 ppm-weight-%.

Preferably, the solid material W is a waste solid material.

Preferably the waste material is one or more of an end-of-life material, such as an end-of-life foam, end-of-life flexible foam, end-of-life rigid foam, an end-of-life compact elastomer, and end-of-life compact duromer. In the context of the present invention, an end-of-life material is a material at the end of the product lifecycle.

Preferably the solid material W comprises, in addition to the one or more polymers, impurities which can be one or more of glass, sand, wood, metals, papers, inorganic solids and polymers other than polyurethanes, polyurethane ureas, polyisocyanurates. The polymers other than polyurethane, polyurethane urea, polyisocyanurate can be for example one or more of polyethylene (PE), polypropylene (PP), polyethylene terephthalate (PET) or polystyrene (PS).

Preferably, the solid material W is one or more of a powder, pieces of foam, pellets, granulates and flakes, more preferably one or more of a powder, pellets, granulates and flakes.

Preferably the average particle size of the solid material W provided in (i) is in the range of from 10⁻⁶ to 10⁻¹ m, preferably in the range of from 10⁻⁶ to 10⁻² m, the average particle size being determined by laser diffraction or light microscopy. These methods being adapted by the skilled person depending on the particle size range. Thus, particle sieve analysis could also be used for determining the average particle size.

Preferably, the one or more polymers contained in the solid material W are one or more polyurethanes, more preferably the one or more polyurethanes are thermosets or elastomers.

According to the present invention, there is no particular restriction as to the polyurethanes of W which are used in the process of the present invention. However, preferably, the polyurethanes of W are prepared by a process wherein polyisocyanates are reacted with polyols in the presence of catalyst(s) as well-known in the art.

Suitable polyisocyanate components used for the production of the polyurethanes of W comprise any of the polyisocyanates known for the production of polyurethanes. These comprise the aliphatic, cycloaliphatic, and aromatic difunctional or polyfunctional isocyanates known from the prior art, and also any desired mixtures thereof. Examples are diphenylmethane 2,2′-, 2,4′-, and 4,4′-diisocyanate, the mixtures of monomeric diphenylmethane diisocyanates with diphenylmethane diisocyanate homologs having a larger number of rings (polymer MDI), isophorone diisocyanate (IPDI) and its oligomers, tolylene 2,4- and 2,6-diisocyanate (TDI), and mixtures of these, tetramethylene diisocyanate and its oligomers, hexamethylene diisocyanate (HDI) and its oligomers, naphthylene diisocyanate (NDI), and mixtures thereof.

Preferably, tolylene 2,4- and/or 2,6-diisocynate (TDI) or a mixture thereof, monomeric diphenylmethane diisocyanates, and/or diphenylmethane diisocyanate homologs having a larger number of rings (polymer MDI), and mixtures of these. Other possible isocyanates are mentioned by way of example in “Kunststoffhandbuch [Plastics handbook], volume 7, Polyurethane [Polyurethanes]”, Carl Hanser Verlag, 3rd edition 1993, chapter 3.2 and 3.3.2.

Suitable polyols used for the production of the polyurethanes of W are selected from the group consisting of polyether polyols, polyester polyols, polyetherester polyols and mixtures thereof.

Polyetherols are by way of example produced from epoxides, for example propylene oxide and/or ethylene oxide, or from tetrahydrofuran with starter compounds exhibiting hydrogen-activity, for example aliphatic alcohols, phenols, amines, carboxylic acids, water, or compounds based on natural substances, for example sucrose, sorbitol or mannitol, with use of a catalyst. Mention may be made here of basic catalysts and double-metal cyanide catalysts, as described by way of example in WO 2006/034800, EP 0090444, or WO 2005/090440.

Polyesterols are by way of example produced from aliphatic or aromatic dicarboxylic acids and polyhydric alcohols, polythioether polyols, polyesteramides, hydroxylated polyacetals, and/or hydroxylated aliphatic polycarbonates, preferably in the presence of an esterification catalyst. Other possible polyols are mentioned by way of example in “Kunststoffhandbuch [Plastics handbook], volume 7, Polyurethane [Polyurethanes]”, Carl Hanser Verlag, 3rd edition 1993, chapter 3.1.

Alternatively, preferably, the one or more polymers contained in the solid material W are one or more polyurethane ureas. According to the present invention, there is no particular restriction as to the polyurethane ureas which are used in the process of the present invention. However, preferably, the polyurethane ureas of W are prepared by a process wherein polyisocyanates are reacted with polyols in the presence of catalysts as well-known in the art. Suitable polyisocyanates and suitable polyols, preferably polyether polyols, are as defined in the foregoing and known in the art.

Alternatively, preferably, the one or more polymers contained in the solid material W are one or more polyisocyanurates. According to the present invention, there is no particular restriction as to the polyisocyanurates which are used in the process of the present invention. However, preferably, the polyisocyanurates of W are prepared by a process wherein polyisocyanates are reacted with polyols in the presence of at least one catalyst as well-known in the art. Suitable polyisocyanates are those listed above. Other possible isocyanates are mentioned by way of example in “Kunststoffhandbuch [Plastics handbook], volume 7, Polyurethane [Polyurethanes]”, Carl Hanser Verlag, 3rd edition 1993, chapter 3.2 and 3.3.2. Preferably, polyisocyanates are tolylene 2,4- and/or 2,6-diisocynate (TDI) or a mixture thereof, monomeric diphenylmethane diisocyanates, and/or diphenylmethane diisocyanate homologs having a larger number of rings (polymer MDI), and mixtures of these. Suitable polyols used for the production of the polyisocyanurates of W are those known in the art, for example those listed “Kunststoffhandbuch [Plastics handbook], volume 7, Polyurethane [Polyurethanes]”, Carl Hanser Verlag, 3rd edition 1993, chapter 3.2 and 3.3.2. Preferably the polyols used for the production of the polyisocyanurates of W are polyester polyol such as a branched polyester polyol based on terephthalic acid and with OH-number of 245 mg KOH/g.

In the context of the present invention, it is also conceivable as a further alternative that preferably the one or more polymers contained in the solid material W are a mixture of one or more of polyurethanes, polyisocyanurates and polyurethane ureas.

Preferably, the reactor unit R_{A} comprises, more preferably consists of, one or more reactors, more preferably at least two reactors, more preferably two reactors, wherein preferably the at least two reactors are arranged in parallel.

Preferably, each of the one or more reactors is a stirred reactor, preferably a stirred tank reactor.

Preferably, each of the one or more reactors is a heated reactor, an adiabatic reactor or an autoclave.

Preferably, the one or more primary amines used in (i.2) are free of hydroxyl groups.

Preferably the one or more primary amines used in (i.2) are free of hydroxyl groups and free of aldehyde groups. More preferably the one or more primary amines used in (i.2) are free of hydroxyl groups, free of aldehyde groups and free of ketone groups.

In other words, preferably the atoms forming the one or more primary amines used in (i.2) are C, H and N.

Preferably, the one or more primary amines used in (i.2), as the first solvent, are selected from the group consisting of aliphatic monoamines, aliphatic polyamines, aromatic monoamines, aromatic polyamines, and mixtures of two or more thereof, more preferably from the group consisting of aliphatic monoamines, aliphatic polyamines, aromatic monoamines, and mixtures of two or more thereof, more preferably from the group consisting of aliphatic monoamines, aromatic monoamines, and mixtures of two thereof.

More preferably, the one or more primary amines are aliphatic monoamines having the formula H₂NR¹, wherein R¹ is selected from the group consisting of (C₃-C₂₅)alkyl, phenyl, (C₇-C₂₅) aralkyl, and (C₇-C₂₅)alkaryl, preferably from the group consisting of (C₃-C₂₀)alkyl, (C₈-C₂₂) aralkyl, and (C₈-C₂₂)alkaryl, more preferably from the group consisting of (C₃-C₁₀)alkyl, phenyl, (C₁₀-C₂₀) aralkyl, and (C₁₀-C₂₀)alkaryl. More preferably the aliphatic monoamines are selected from the group consisting of n-butylamines, cyclohexylamines, n-octylamines, n-hexylamines, n-propylamines, n-dodecylamines, n-tridecylamines, n-octadecylamines, and mixtures of two or more thereof, more preferably selected from the group consisting of n-butylamines, n-octylamines, n-hexylamines and n-propylamines, wherein more preferably the aliphatic monoamines are n-butylamines.

Alternatively, more preferably, the one or more primary amines are aromatic monoamines, wherein the aromatic monoamines are selected from the group consisting of aniline, toluidine, naphtylamine, and mixtures of two or more thereof, wherein the aromatic monoamines more preferably are aniline.

Alternatively, more preferably, the one or more primary amines are aliphatic polyamines, wherein the aliphatic polyamines are selected from the group consisting of hexamethylendiamine, ethylenediamine, propanediamine, such as propane-1,3-diamine, propane-1,2-diamine, isophorone diamine, butanediamine, such as butane-1,4-diamine, butane-1,3-diamine, pentadiamine, such as pentane-1,5-diamine, diaminocyclohexane, such as 1,2-diaminocyclohexane, and a mixture of two or more thereof, more preferably selected from the group consisting of hexamethylendiamine, ethylenediamine, propanediamine and butanediamine, more preferably selected from the group consisting of hexamethylendiamine, ethylenediamine, and propanediamine. More preferably, the aliphatic polyamines are selected from the group consisting of ethylendiamine and propanediamines.

In the context of the present invention, all isomers of the aforementioned aliphatic polyamines can be envisaged. Preferably, propanediamine is propane-1,3-diamine or propane-1,2-diamine.

Alternatively, more preferably, the one or more primary amines are aromatic polyamines having the formula H₂N—R²—NH₂,
wherein R² is selected from the group consisting of (C₃-C₂₅)alkylene, (C₆-C₂₅) aralkylene, and (C₆-C₂₅)alkarylene, preferably from the group consisting of (C₈-C₂₂)alkylene, (C₈-C₂₂) aralkylene, and (C₈-C₂₂)alkarylene, more preferably from the group consisting of (C₁₀-C₂₀)alkylene, (C₁₀-C₂₀) aralkylene, and (C₁₀-C₂₀)alkarylene. More preferably the aromatic polyamines are selected from the group consisting of diaminotoluene, phenylenediamine and diaminodiphenylmethane, more preferably selected from the group consisting of diaminotoluene, phenylenediamine and 4,4′-diaminodiphenylmethane, more preferably is diaminotoluene, more preferably 2,4-diaminotoluene.

In the context of the present invention, more preferably, the one or more primary amines are aromatic monoamines or aliphatic monoamines. More preferably, the one or more primary amines used in (i.2) are aniline or n-butylamine.

Preferably, the ratio of the weight of the solid material W introduced into R_{A} relative to the weight of the one or more primary amines introduced into R_{A} is in the range of from 1:100 to 1:1, more preferably in the range of from 1:40 to 1:3, more preferably in the range of from 1:10 to 1:5.

Preferably, the aminolysis reaction according to (i.3) is performed at a temperature in the range of from 50 to 250° C., more preferably in the range of from 80 to 200° C., more preferably in the range of from 100 to 220° C., more preferably in the range of from 140 to 200° C.

Preferably, the aminolysis reaction according to (i.3) is performed at a pressure in the range of from 1.0 to 25.0 bar (abs), more preferably in the range of from 1.0 to 20.0 bar (abs), more preferably in the range of from 1.0 to 18.0 bar (abs).

Preferably, the aminolysis reaction according to (i.3) is conducted for a duration in the range of from 1 to 600 min, more preferably in the range of from 20 to 450 min, more preferably in the range of from 60 to 300 min.

Preferably, at most 0.1 weight-%, more preferably from 0 to 0.01 weight-%, more preferably from 0 to 0.001 weight-%, of the mixture M obtained according to (i) consist of polymer selected from the group consisting of polyurethane, polyurethane urea and polyisocyanurate.

In other words, it is preferred that the mixture M is substantially free of, more preferably free of, polymer selected from the group consisting of polyurethane, and polyisocyanurate, meaning essentially free of, more preferably free of polyurethane, and of polyisocyanurate. This is identified by the analysis of the mixture M by IR spectroscopy: The mixture M presents no NCO-stretching vibration at 2200-2400 cm⁻¹ meaning that the reaction (aminolysis) is complete.

Preferably, the process further comprises, after (i.4) and prior to (ii), passing the mixture M removed from R_{A} according to (i.4) into a solid-liquid separation unit, obtaining a liquid mixture M_{SLS} comprising the one or more polyurea-containing compounds and the one or more polyols and a solid mixture comprising impurities.

Preferably the impurities being one or more of glass, sand, wood, metals, papers, inorganic solids and polymers other than polyurethanes, polyurethane ureas, polyisocyanurates. The polymers other than polyurethane, polyurethane urea, polyisocyanurate can be for example one or more of polyethylene (PE), polypropylene (PP), polyethylene terephthalate (PET) or polystyrene (PS).

Preferably, the solid-liquid separation unit is a filtration unit or a centrifuge, more preferably a filtration unit, more preferably a filter, more preferably a pocket filter, a bag filter, a membrane filter, a candle filter, an agitated pressure filter, a vacuum belt filter, a frame & plate filter, or a nutsche filter.

Preferably, passing the mixture M removed from R_{A} according to (i.4) into a solid-liquid separation unit is performed at a temperature in the range of from 50 to 250° C., more preferably in the range of from 80 to 200° C., more preferably in the range of from 100 to 220° C., more preferably in the range of from 140 to 200° C.

Preferably, the solid-liquid separation is performed at a pressure 1.0 to 20.0 bar (abs), more preferably in the range of from 1.0 to 20.0 bar (abs), more preferably in the range of from 1.0 to 18.0 bar (abs).

Preferably, (ii) comprises
passing a second solvent S and the liquid mixture M_{SLS} into an evaporation unit EU, obtaining, from the evaporation unit EU, a vapor mixture M_{V} comprising S and at least a portion of the one or more primary amines, and a liquid mixture M_{L} comprising the one or more polyurea-containing compounds, and the one or more polyols.

Preferably S has a water content in the range of from 0 to 1000 ppm, more preferably in the range of from 0 to 500 ppm, more preferably in the range of from 0 to 100 ppm. In the context of the present invention, said solvent is thus preferably an anhydrous solvent.

Preferably, S used in (ii) is selected from the group consisting of hydrocarbons, ketones and ether, more preferably selected from the group consisting of xylene, toluene, n-heptane, methyl ethyl ketone (MEK), dioxane, benzene, heptan-2-one and a mixture of two or more thereof, more preferably is selected from the group consisting of xylene, toluene, n-heptane, benzene and a mixture of two or more thereof, more preferably is xylene or toluene or benzene.

Preferably, for (ii), the amount of S added is calculated on the basis of the weight ratio of said second solvent S relative to the one or more polymers of W provided according (i.1) being in the range of from 0.1:1 to 50:1, more preferably in the range of from 1:1 to 20:1, more preferably in the range of from 1:1 to 15:1, more preferably in the range of from 1:1 to 10:1.

Preferably the evaporation unit EU used in (ii) is one or more of a reactor equipped with a filter, a fractioned distillation column and a flash drum, more preferably a reactor equipped with a filter, more preferably a stirred tank reactor equipped with a filter.

Preferably, the evaporation in EU according to (ii) is performed at a pressure difference, being the difference between the pressure before the evaporation unit and the pressure at the evaporation unit, being in the range from 0 to 30 bar (abs), more preferably in the range of from 0.1 to 25 bar (abs), more preferably in the range of from 0.2 to 20 bar (abs).

Preferably, the liquid mixture M_{L} comprises at most 10 weight-%, more preferably at most 8 weight-%, more preferably at most 5 weight-%, of the one or more primary amines, based on the weight of the liquid mixture M_{L}.

Preferably, (iii) comprises
(iii.1) adding a solvent, more preferably S, to the liquid mixture M_{L} obtained according to (ii), allowing the one or more polyurea-containing compounds to precipitate, obtaining a mixture comprising one or more precipitated polyurea-containing compounds;
(iii.2) passing the mixture comprising the one or more precipitated polyurea-containing compounds obtained according to (iii.1) through a solid-liquid separation unit SLU, obtaining a liquid mixture P comprising the one or more polyols, and a solid mixture U comprising the one or more polyurea-containing compounds.

Preferably, the present invention relates to a solvent-efficient process for recycling one or more polymers, selected from the group consisting of polyurethanes, polyurethane ureas, polyisocyanurates, and a mixture of two or more thereof, said one or more polymers being contained in a solid material W, the process comprising
(i) preparing a mixture M comprising one or more polyurea-containing compounds, and further comprising one or more polyols, comprising:
   (i.1) providing a solid material W comprising one or more polymers selected from the group consisting of polyurethanes, polyurethane ureas, polyisocyanurates, and a mixture of two or more thereof;
   (i.2) introducing the solid material W provided in (i.1) and one or more primary amines, as a first solvent, in a reactor unit R_{A}, obtaining a mixture;
   (i.3) subjecting the mixture obtained according to (i.2) in R_{A} to aminolysis reaction conditions, obtaining a mixture M comprising one or more polyurea-containing compounds, and further comprising one or more polyols and one or more primary amines;
   (i.4) removing the mixture M obtained according to (i.3) from R_{A};
(ii) passing a second solvent S and the mixture M obtained according to (i) into an evaporation unit EU, obtaining, from the evaporation unit EU, a vapor mixture M_{V} comprising S and at least a portion of the one or more primary amines, and a liquid mixture M_{L} comprising the one or more polyurea-containing compounds, and the one or more polyols;
(iii) isolating the one or more polyurea-containing compounds from M_{L} obtained according to (ii), wherein (iii) comprises
   (iii.1) adding a solvent, more preferably S, to the liquid mixture M_{L} obtained according to (ii), allowing the one or more polyurea-containing compounds to precipitate, obtaining a mixture comprising one or more precipitated polyurea-containing compounds;
   (iii.2) passing the mixture comprising the one or more precipitated polyurea-containing compounds obtained according to (iii.1) through a solid-liquid separation unit SLU, obtaining a liquid mixture P comprising the one or more polyols, and a solid mixture U comprising the one or more polyurea-containing compounds;
(iv) subjecting the mixture U comprising the one or more polyurea-containing compounds obtained according to (iii) to cleavage reaction conditions in a reaction unit R_{C}, obtaining either one or more corresponding polyamines or one or more corresponding polyisocyanates;
(v) separating the one or more primary amines from M_{V} obtained according to (ii) in a purification unit PU, obtaining a vapor mixture A comprising the one or more primary amines depleted in S;
(vi) recycling at least a part of A obtained according to (v) from PU into R_{A} in (i.2) as the first solvent. More preferably, the present invention relates to a solvent-efficient process for recycling one or more polymers, selected from the group consisting of polyurethanes, polyurethane ureas, polyisocyanurates, and a mixture of two or more thereof, said one or more polymers being contained in a solid material W, the process comprising
(i) preparing a mixture M comprising one or more polyurea-containing compounds, and further comprising one or more polyols, comprising:
   (i.1) providing a solid material W comprising one or more polymers selected from the group consisting of polyurethanes, polyurethane ureas, polyisocyanurates, and a mixture of two or more thereof;
   (i.2) introducing the solid material W provided in (i.1) and one or more primary amines, as a first solvent, in a reactor unit R_{A}, obtaining a mixture, wherein the atoms forming the one or more primary amines are C, H and N;
   (i.3) subjecting the mixture obtained according to (i.2) in R_{A} to aminolysis reaction conditions, obtaining a mixture M comprising one or more polyurea-containing compounds, and further comprising one or more polyols and one or more primary amines;
   (i.4) removing the mixture M obtained according to (i.3) from R_{A};
(ii) passing a second solvent S and the mixture M obtained according to (i) into an evaporation unit EU, wherein S has a water content in the range of from 0 to 1000 ppm, more preferably in the range of from 0 to 500 ppm, more preferably in the range of from 0 to 100 ppm, obtaining, from the evaporation unit EU, a vapor mixture M_{V} comprising S and at least a portion of the one or more primary amines, and a liquid mixture M_{L} comprising the one or more polyurea-containing compounds, and the one or more polyols;
(iii) isolating the one or more polyurea-containing compounds from M_{L} obtained according to (ii), wherein (iii) comprises
   (iii.1) adding a solvent, more preferably S, to the liquid mixture M_{L} obtained according to (ii), allowing the one or more polyurea-containing compounds to precipitate, obtaining a mixture comprising one or more precipitated polyurea-containing compounds;
   (iii.2) passing the mixture comprising the one or more precipitated polyurea-containing compounds obtained according to (iii.1) through a solid-liquid separation unit SLU, obtaining a liquid mixture P comprising the one or more polyols, and a solid mixture U comprising the one or more polyurea-containing compounds;
(iv) subjecting the mixture U comprising the one or more polyurea-containing compounds obtained according to (iii) to cleavage reaction conditions in a reaction unit R_{C}, obtaining either one or more corresponding polyamines or one or more corresponding polyisocyanates;
(v) separating the one or more primary amines from M_{V} obtained according to (ii) in a purification unit PU, obtaining a vapor mixture A comprising the one or more primary amines depleted in S;
(vi) recycling at least a part of A obtained according to (v) from PU into R_{A} in (i.2) as the first solvent.

In the context of the present invention, preferably, the solvent used in (iii. 1) is selected from the group consisting of hydrocarbons, ketones and ether, more preferably selected from the group consisting of xylene, toluene, n-heptane, methyl ethyl ketone (MEK), dioxane, benzene, heptan-2-one and a mixture of two or more thereof, preferably is selected from the group consisting of xylene, toluene, n-heptane, benzene and a mixture of two or more thereof, more preferably is xylene or toluene or benzene.

Preferably the solvent used in (iii) is the same as used in (ii), namely S.

Preferably, for (iii. 1), the amount of the solvent added is calculated on the basis of the weight ratio of said solvent relative to the one or more polymers of W provided according (i.1) being in the range of from 0.1:1 to 50:1, more preferably in the range of from 1:1 to 20:1, more preferably in the range of from 1:1 to 15:1, more preferably in the range of from 1:1 to 10:1.

Preferably, the solid-liquid separation unit SLU in (iii.2) is a filter.

Preferably at most 0.1 weight-%, preferably from 0 to 0.01 weight-%, more preferably from 0 to 0.001 weight-%, of the mixture U consist of polyol.

In other words, it is preferred that the mixture U be substantially free of, more preferably free of, polyol. This can be for example identified with ¹³C_NMR or HPLC.

Preferably, the process further comprises passing the mixture P comprising the one or more polyols obtained according to (iii.2) into a storage tank T4.

In the context of the present invention, the one or more polyols obtained as described above may be employed for every conceivable use in industrial and consumer applications, including, but not limited to, polyurethane compounds which, in turn, may be employed for uses including, but not limited to:
Foam cushioning, for example for furniture, bedding, and packaging
Rigid foam insulation, for example for building and refrigeration
Flexible foam, for example for upholstery, mattrasses, footwear, and seals
Elastomers, for example for automotive and industrial parts, such as suspension bushings, gaskets, and seals
Adhesives, sealants, and coatings
Roller and drive belts, for example for conveyors, power transmissions, and agricultural equipment
Medical devices and implants, such as prosthetics and artificial heart valves
Fibers, non-woven fabrics, and textiles
Paints, coatings, and inks
Electronic components, such as casings, gaskets, and vibration dampers.

Preferably, the process further comprises, after (iii) and prior to (iv), washing the mixture U obtained according to (ii) with a solvent on SLU, more preferably the same solvent as used in (iii. 1), more preferably S. Preferably said solvent is selected from the group consisting of hydrocarbons, ketones and ether, more preferably selected from the group consisting of benzene, xylene, toluene, n-heptane, methyl ethyl ketone (MEK), dioxane, heptan-2-one and a mixture of two or more thereof, preferably is selected from the group consisting of xylene, toluene, n-heptane, benzene, and a mixture of two or more thereof, more preferably is benzene, xylene or toluene.

Preferably said solvent has a water content in the range of from 0 to 1000 ppm, more preferably in the range of from 0 to 500 ppm, more preferably in the range of from 0 to 100 ppm. In the context of the present invention, said solvent is thus preferably an anhydrous solvent.

Preferably the ratio of the weight of the solvent relative to the weight of the solid mixture U is in the range of from 0.01:1 to 100:1, more preferably in the range of from 0.1:1 to 10:1, more preferably in the range of from 1:1 to 5:1.

Preferably, the process further comprises passing the filtrate obtained after the washing of U into the storage tank T4.

Preferably the reactor unit R_{C} comprises one or more reactors, more preferably at least two reactors, more preferably in the range of from 2 to 5 reactors, more preferably two reactors, the at least two reactors being more preferably arranged in parallel.

Preferably R_{C} is one or more of the evaporation unit EU and the solid-liquid separation unit SLU, preferably is the evaporation unit EU and the solid-liquid separation unit SLU. Hence, preferably (ii), (iv) and (v) are performed in the same unit, said unit being more preferably a stirred tank reactor equipped with a filter. Such configuration is possible if it is a batch process.

Alternatively, it is preferred that R_{C}, EU and SLU be three distinctive units, wherein EU is located upstream of SLU which is located upstream of Rc.

Preferably, the cleavage reaction conditions according to (iv) are hydrolysis reaction conditions.

Preferably, (iv) comprises
admixing water with the mixture U comprising the one or more polyurea-containing compounds obtained according to (iii) in R_{C}, wherein the weight ratio of water relative to the one or more polyurea-containing compounds is in the range of from 1:1 to 50:1, more preferably in the range of from 5:1 to 20:1, more preferably in the range of from 10:1 to 18:1, obtaining one or more corresponding polyamines.

Preferably, the hydrolysis is performed at a temperature in the range of from 150 to 350° C., more preferably in the range of from 210 to 290° C., more preferably in the range of from 230 to 270° C.

Preferably, the hydrolysis is performed for a duration in the range of from 0.1 to 15 h, more preferably in the range of from 0.25 to 10 h, more preferably in the range of from 0.5 to 8 h.

Preferably, the hydrolysis is performed at a pressure in the range of from 20 to 70 bar (abs), more preferably in the range of from 25 to 65 bar (abs), more preferably in the range of from 30 to 60 bar (abs).

Alternatively, preferably, the cleavage reaction conditions according to (iv) are acidic cleavage conditions. More preferably, (iv) comprises
admixing a Bronsted acid, wherein more preferably the Bronsted acid is selected from the group consisting of hydrochloric acid, a sulfonic acid and a mixture thereof, with the mixture U comprising the one or more polyurea-containing compounds obtained according to (iii) in R_{C}, obtaining one or more corresponding polyisocyanates.

In the context of the present invention, the one or more polyisocyanates obtained as described above may be employed for every conceivable use in industrial and consumer applications, including, but not limited to, polyurethane compounds which, in turn, may be employed for uses including, but not limited to:
Foam cushioning, for example for furniture, bedding, and packaging
Rigid foam insulation, for example for building and refrigeration
Flexible foam, for example for upholstery, mattrasses, footwear, and seals
Elastomers, for example for automotive and industrial parts, such as suspension bushings, gaskets, and seals
Adhesives, sealants, and coatings
Roller and drive belts, for example for conveyors, power transmissions, and agricultural equipment
Medical devices and implants, such as prosthetics and artificial heart valves
Fibers, non-woven fabrics, and textiles
Paints, coatings, and inks
Electronic components, such as casings, gaskets, and vibration dampers.

Preferably the sulfonic acid is one or more of methane sulfonic acid, toluene sulfonic acid and trifluromethane sulfonic acid.

Alternatively, preferably, the cleavage reaction conditions according to (iv) are basic cleavage conditions. More preferably, (iv) comprises
admixing a Bronsted base with the solid mixture U comprising the one or more polyurea-containing compounds obtained according to (iii) in R_{C}, wherein the Bronsted base has a pH value in water in the range of from 8 to 12, obtaining a mixture comprising one or more corresponding polyisocyanates.

Examples of the base for the cleavage reactions are alkali hydroxides, alkaline earth hydroxides, 1,8-Diazabicyclo[5.4.0]undec-7-ene (DBU), 1,4-diazabicyclo[2.2.2]octane (DABCO), triethylamine and primary amines as defined in the foregoing for the aminolysis reaction.

Preferably, (iv) comprises
(iv.1) subjecting the mixture U comprising the one or more polyurea-containing compounds obtained according to (iii) to cleavage reaction conditions, more preferably hydrolysis reaction conditions, obtaining a mixture comprising one or more corresponding polyamines;
(iv.2) passing the mixture obtained according to (iv.1) through an evaporation unit EU1, obtaining from EU1 a vapor mixture M_{V1} comprising water and at least a portion of the one or more primary amines, and a liquid mixture M_{EU1} comprising the one or more polyamines;
(iv.3) passing the vapor mixture M_{V1} obtained according to (iv.2) through a heat exchanger for condensation, obtaining a liquid mixture comprising water and at least a portion of the one or more primary amines, more preferably passing said obtained liquid mixture into a tank unit or a stripping column for removal of carbon dioxide;
(iv.4) passing the liquid mixture obtained according to (iv.3) in PU used according to (v), obtaining a vapor mixture comprising the one or more primary amines depleted in water;
(iv.5) recycling at least a part of the vapor mixture obtained according to (iv.4) from PU into R_{A} in (i.2) as the first solvent.

Preferably, the evaporation unit EU1 used in (iv.2) is one or more of a flash tank and a stripping column, more preferably a flash tank or a stripping column.

Preferably, the process further comprises
passing the liquid mixture M_{EU1} comprising the one or more polyamines obtained according to (iv.2) in a heat exchanger, and introducing the obtained mixture in a decanter D, obtaining a lower phase comprising the one or more polyamines and an upper phase comprising water and at least a portion of the one or more primary amines;
more preferably passing the upper phase through a compressor and subsequently the purification unit PU, more preferably PU2 as defined below.

Preferably, the process further comprises
introducing the lower phase into a purification unit PUA, more preferably a distillation column, obtaining a liquid mixture comprising the one or more polyamines, wherein more preferably from 99 to 100 weight-%, more preferably from 99.5 to 100 weight-%, more preferably from 99.9 to 100 weight-%, of said mixture consist of the one or more polyamines and obtaining a vapor mixture comprising at least a portion of the one or more primary amines;
more preferably introducing the vapor mixture into PU, more preferably PU2 as defined below.

Preferably, prior to (v), the process further comprises storing M_{V} obtained according to (ii) in a storage tank T3.

Preferably, the purification unit PU comprises a first purification sub-unit PU1 and a second purification sub-unit PU2, more preferably PU1 and PU2 are serially coupled sub-units. Preferably, the purification sub-unit PU1 is a distillation column. Preferably, the purification sub-unit PU2 is a distillation column.

Preferably, (v) comprises
(v.1) introducing M_{V} obtained according to (ii) into PU1, obtaining a vapor mixture A1 comprising the one or more primary amines and a liquid mixture comprising S;
(v.2) introducing the vapor mixture A1 obtained according to (v.1) into PU2, obtaining a vapor mixture A comprising the one or more primary amines, wherein the concentration of primary amines in A is greater than the concentration of the primary amines in A1.

Preferably, the process further comprises
storing the liquid mixture comprising S obtained according to (v.1) in a storage tank T2 prior to be recycled and use as the solvent in (ii) and/or (iii. 1), more preferably in (ii) and (iii. 1).

Preferably, (iv) comprises
(iv.1) subjecting the mixture U comprising the one or more polyurea-containing compounds obtained according to (iii) to cleavage reaction conditions, obtaining a mixture comprising one or more corresponding polyamines;
(iv.2) passing the mixture obtained according to (iv.1) in an evaporation unit EU1, obtaining from EU1 a vapor mixture M_{V1} comprising water and at least a portion of the one or more primary amines, and a liquid mixture comprising the one or more polyamines;
(iv.3) passing the vapor mixture M_{V1} obtained according to (iv.2) through a heat exchanger for condensation, obtaining a liquid mixture comprising water and at least a portion of the one or more primary amines, preferably passing said obtained liquid mixture into a tank unit or a stripping column for removal of carbon dioxide;
(iv.4) passing the liquid mixture obtained according to (iv.3) in PU2 used according to (v), obtaining a vapor mixture comprising the one or more primary amines depleted in water;
(iv.5) recycling at least a part of the vapor mixture obtained according to (iv.4) from PU2 into R_{A} in (i.2) as the first solvent.

Preferably, the process further comprises
passing the mixture P obtained according to (iii) in a purification unit PUP for separating the one or more polyols from the liquid mixture P, obtaining a mixture, comprising the one or more poylols, depleted from at least a portion of compounds other than polyols.

Alternatively, preferably, (iv) comprises
passing the liquid mixture P obtained according to (ii) in a purification unit PUP, obtaining a liquid mixture, comprising one or more polyols, depleted from at least a portion of the solvent S and a vapor mixture Vs comprising at least a portion S. Preferably, the purification unit PUP comprises, more preferably is, a distillation column.

More preferably, (iv) further comprises
passing the vapor mixture Vs comprising at least a portion of the solvent S and water in a purification unit PU1, obtaining a liquid mixture comprising the solvent;
introducing the obtained liquid mixture in a storage tank T2, more preferably prior to be used as S in (ii.1) and/or (ii.2).

Preferably, the process is a semi-batch process.

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”. Further, it is explicitly noted that the following set of embodiments represents a suitably structured part of the general description directed to preferred aspects of the present invention, and, thus, suitably supports, but does not represent the claims of the present invention.
1. A solvent-efficient process for recycling one or more polymers, selected from the group consisting of polyurethanes, polyurethane ureas, polyisocyanurates, and a mixture of two or more thereof, said one or more polymers being contained in a solid material W, the process comprising
   (i) preparing a mixture M comprising one or more polyurea-containing compounds, and further comprising one or more polyols, comprising:
      (i.1) providing a solid material W comprising one or more polymers selected from the group consisting of polyurethanes, polyurethane ureas, polyisocyanurates, and a mixture of two or more thereof;
      (i.2) introducing the solid material W provided in (i.1) and one or more primary amines, as a first solvent, in a reactor unit R_{A}, obtaining a mixture;
      (i.3) subjecting the mixture obtained according to (i.2) in R_{A} to aminolysis reaction conditions, obtaining a mixture M comprising one or more polyurea-containing compounds, and further comprising one or more polyols and one or more primary amines;
      (i.4) removing the mixture M obtained according to (i.3) from R_{A};
   (ii) passing a second solvent S and the mixture M obtained according to (i) into an evaporation unit EU, obtaining, from the evaporation unit EU, a vapor mixture M_{V} comprising S and at least a portion of the one or more primary amines, and a liquid mixture M_{L} comprising the one or more polyurea-containing compounds, and the one or more polyols;
   (iii) isolating the one or more polyurea-containing compounds from M_{L} obtained according to (ii), obtaining a mixture U comprising the one or more polyurea-containing compounds and a mixture P comprising the one or more polyols;
   (iv) subjecting the mixture U comprising the one or more polyurea-containing compounds obtained according to (iii) to cleavage reaction conditions in a reaction unit R_{C}, obtaining either one or more corresponding polyamines or one or more corresponding polyisocyanates;
   (v) separating the one or more primary amines from M_{V} obtained according to (ii) in a purification unit PU, obtaining a vapor mixture A comprising the one or more primary amines depleted in S;
   (vi) recycling at least a part of A obtained according to (v) from PU into R_{A} in (i.2) as the first solvent.
2. The process of embodiment 1, further comprising, prior to (i), subjecting the solid material W to a pre-treatment, preferably a mechanical pre-treatment, wherein the mechanical pre-treatment preferably comprises one or more of milling, crushing, shredding and cutting, preferably milling or shredding, of the solid material W.
3. The process of embodiment 1 or 2, further comprising, prior to (i), preferably after subjecting the solid material W to a pre-treatment as defined in embodiment 2, drying the solid material W, wherein drying is preferably conducted at a temperature in the range of from 40 to 100° C., more preferably in the range of from 50 to 85° C., and wherein more preferably drying is conducted in a gas atmosphere comprising one or more of nitrogen and oxygen, more preferably air.
4. The process of any one of embodiments 1 to 3, wherein the solid material W is a waste solid material.
5. The process of any one of embodiments 1 to 4, wherein the one or more polymers contained in the solid material W are one or more polyurethanes, preferably the one or more polyurethanes being thermosets or elastomers.
6. The process of any one of embodiments 1 to 4, wherein the one or more polymers contained in the solid material W are one or more polyurethane ureas.
7. The process of any one of embodiments 1 to 4, wherein the one or more polymers contained in the solid material W are one or more polyisocyanurates.
8. The process of any one of embodiments 1 to 7, wherein the reactor unit R_{A} comprises, preferably consists of, one or more reactors, preferably at least two reactors, more preferably two reactors, wherein preferably the at least two reactors are arranged in parallel.
9. The process of embodiment 8, wherein each of the one or more reactors is a stirred reactor, preferably a stirred tank reactor.
10. The process of embodiment 8 or 9, wherein each of the one or more reactors is a heated reactor, an adiabatic reactor or an autoclave.
11. The process of any one of embodiments 1 to 10, wherein the one or more primary amines used in (i.2) are free of hydroxyl groups, preferably wherein the atoms forming the one or more primary amines used in (i.2) are C, H and N.
12. The process of any one of embodiments 1 to 11, wherein the one or more primary amines used in (i.2), as the first solvent, are selected from the group consisting of aliphatic monoamines, aliphatic polyamines, aromatic monoamines, aromatic polyamines, and mixtures of two or more thereof, preferably from the group consisting of aliphatic monoamines, aliphatic polyamines, aromatic monoamines, and mixtures of two or more thereof, more preferably from the group consisting of aliphatic monoamines, aromatic monoamines, and mixtures of two thereof.
13. The process of embodiment 12, wherein the one or more primary amines are aliphatic monoamines having the formula H₂NR¹,
wherein R¹ is selected from the group consisting of (C₃-C₂₅)alkyl, phenyl, (C₇-C₂₅) aralkyl, and (C₇-C₂₅)alkaryl, preferably from the group consisting of (C₃-C₂₀)alkyl, (C₈-C₂₂) aralkyl, and (C₈-C₂₂)alkaryl, more preferably from the group consisting of (C₃-C₁₀)alkyl, phenyl, (C₁₀-C₂₀) aralkyl, and (C₁₀-C₂₀)alkaryl,
wherein more preferably the aliphatic monoamines are selected from the group consisting of n-butylamines, cyclohexylamines, n-octylamines, n-hexylamines, n-propylamines, n-dodecylamines, n-tridecylamines, n-octadecylamines, and mixtures of two or more thereof, more preferably selected from the group consisting of n-butylamines, n-octylamines, n-hexylamines and n-propylamines, wherein more preferably the aliphatic monoamines are n-butylamines.
14. The process of embodiment 12, wherein the one or more primary amines are aromatic monoamines, wherein the aromatic monoamines are selected from the group consisting of aniline, toluidine, naphtylamine, and mixtures of two or more thereof, wherein the aromatic monoamines preferably are aniline.
15. The process of embodiment 12, wherein the one or more primary amines are aliphatic polyamines, wherein the aliphatic polyamines are selected from the group consisting of hexamethylendiamine, ethylenediamine, propanediamine, isophorone diamine, butanediamine, pentadiamine, diaminocyclohexane and a mixture of two or more thereof, more preferably selected from the group consisting of hexamethylendiamine, ethylenediamine, propanediamine and butanediamine, more preferably selected from the group consisting of hexamethylendiamine, ethylenediamine, and propanediamine; wherein the aliphatic polyamines more preferably are selected from the group consisting of ethylendiamine and propanediamines.
16. The process of embodiment 12, wherein the one or more primary amines are aromatic polyamines having the formula H₂N—R²—NH₂,
wherein R² is selected from the group consisting of (C₃-C₂₅)alkylene, (C₆-C₂₅) aralkylene, and (C₆-C₂₅)alkarylene, preferably from the group consisting of (C₈-C₂₂)alkylene, (C₈-C₂₂) aralkylene, and (C₈-C₂₂)alkarylene, more preferably from the group consisting of (C₁₀-C₂₀)alkylene, (C₁₀-C₂₀) aralkylene, and (C₁₀-C₂₀)alkarylene,
wherein more preferably the aromatic polyamines are selected from the group consisting of diaminotoluene, phenylenediamine and diaminodiphenylmethane, more preferably selected from the group consisting of diaminotoluene, phenylenediamine and 4,4′-diaminodiphenylmethane, more preferably is diaminotoluene, more preferably 2,4-diaminotoluene.
17. The process of any one of embodiments 1 to 16, wherein the ratio of the weight of the solid material W introduced into R_{A} relative to the weight of the one or more primary amines introduced into R_{A} is in the range of from 1:100 to 1:1, preferably in the range of from 1:40 to 1:3, more preferably in the range of from 1:10 to 1:5.
18. The process of any one of embodiments 1 to 17, wherein the aminolysis reaction according to (i.3) is performed at a temperature in the range of from 50 to 250° C., preferably in the range of from 80 to 200° C., more preferably in the range of from 100 to 220° C., more preferably in the range of from 140 to 200° C.
19. The process of any one of embodiments 1 to 18, wherein the aminolysis reaction according to (i.3) is performed at a pressure in the range of from 1.0 to 25.0 bar (abs), preferably in the range of from 1.0 to 20.0 bar (abs), more preferably in the range of from 1.0 to 18.0 bar (abs).
20. The process of any one of embodiments 1 to 19, wherein the aminolysis reaction according to (i.3) is conducted for a duration in the range of from 1 to 600 min, preferably in the range of from 20 to 450 min, more preferably in the range of from 60 to 300 min.
21. The process of any one of embodiments 1 to 20, wherein at most 0.1 weight-%, preferably from 0 to 0.01 weight-%, more preferably from 0 to 0.001 weight-%, of the mixture M obtained according to (i) consist of polymer selected from the group consisting of polyurethane, polyurethane urea and polyisocyanurate.
22. The process of any one of embodiments 1 to 21, further comprising, after (i.4) and prior to (ii), passing the mixture M removed from R_{A} according to (i.4) into a solid-liquid separation unit, obtaining a liquid mixture M_{SLS} comprising the one or more polyurea-containing compounds and the one or more polyols and a solid mixture comprising impurities.
23. The process of embodiment 22, wherein passing the mixture M removed from R_{A} according to (i.4) into a solid-liquid separation unit is performed at a temperature in the range of from 50 to 250° C., preferably in the range of from 80 to 200° C., more preferably in the range of from 100 to 220° C., more preferably in the range of from 140 to 200° C.
24. The process of embodiment 22 or 23, wherein (ii) comprises passing a second solvent S and the liquid mixture M_{SLS} into an evaporation unit EU, obtaining, from the evaporation unit EU, a vapor mixture M_{V} comprising S and at least a portion of the one or more primary amines, and a liquid mixture M_{L} comprising the one or more polyurea-containing compounds, and the one or more polyols.
25. The process of any one of embodiments 1 to 24, wherein S used in (ii) is selected from the group consisting of hydrocarbons, ketones and ether, more preferably selected from the group consisting of xylene, toluene, n-heptane, methyl ethyl ketone (MEK), dioxane, benzene, heptan-2-one and a mixture of two or more thereof, preferably is selected from the group consisting of xylene, toluene, n-heptane, benzene and a mixture of two or more thereof, more preferably is xylene or toluene or benzene; and/or wherein S has a water content in the range of from 0 to 1000 ppm, preferably in the range of from 0 to 500 ppm, more preferably in the range of from 0 to 100 ppm.
26. The process of any one of embodiments 1 to 25, wherein for (ii), the amount of S added is calculated on the basis of the weight ratio of said second solvent S relative to the one or more polymers of W provided according (i.1) being in the range of from 0.1:1 to 50:1, preferably in the range of from 1:1 to 20:1, more preferably in the range of from 1:1 to 15:1, more preferably in the range of from 1:1 to 10:1.
27. The process of any one of embodiments 1 to 26, wherein the evaporation in EU according to (ii) is performed at a pressure difference, being the difference between the pressure before the evaporation unit and the pressure at the evaporation unit, being in the range from 0 to 30 bar (abs), preferably in the range of from 0.1 to 25 bar (abs), more preferably in the range of from 0.2 to 20 bar (abs).
28. The process of any one of embodiments 1 to 27, wherein the liquid mixture M_{L} comprises at most 10 weight-%, preferably at most 8 weight-%, more preferably at most 5 weight-%, of the one or more primary amines, based on the weight of the liquid mixture M_{L}.
29. The process of any one of embodiments 1 to 28, wherein (iii) comprises
   (iii.1) adding a solvent, preferably S, to the liquid mixture M_{L} obtained according to (ii), allowing the one or more polyurea-containing compounds to precipitate, obtaining a mixture comprising one or more precipitated polyurea-containing compounds;
   (iii.2) passing the mixture comprising the one or more precipitated polyurea-containing compounds obtained according to (iii. 1) through a solid-liquid separation unit SLU, obtaining a liquid mixture P comprising the one or more polyols, and a solid mixture U comprising the one or more polyurea-containing compounds.
30. The process of embodiment 29, wherein the solvent used in (iii. 1) is selected from the group consisting of hydrocarbons, ketones and ether, more preferably selected from the group consisting of xylene, toluene, n-heptane, methyl ethyl ketone (MEK), dioxane, benzene, heptan-2-one and a mixture of two or more thereof, preferably is selected from the group consisting of xylene, toluene, n-heptane, benzene and a mixture of two or more thereof, more preferably is xylene or toluene or benzene.
31. The process of embodiment 29 or 30, wherein for (iii. 1), the amount of the solvent added is calculated on the basis of the weight ratio of said solvent relative to the one or more polymers of W provided according (i.1) being in the range of from 0.1:1 to 50:1, preferably in the range of from 1:1 to 20:1, more preferably in the range of from 1:1 to 15:1, more preferably in the range of from 1:1 to 10:1.
32. The process of any one of embodiments 1 to 31, wherein at most 0.1 weight-%, preferably from 0 to 0.01 weight-%, more preferably from 0 to 0.001 weight-%, of the mixture U consist of polyol.
33. The process of any one of embodiments 1 to 32, wherein the cleavage reaction conditions according to (iv) are hydrolysis reaction conditions.
34. The process of embodiment 33, wherein (iv) comprises
   admixing water with the mixture U comprising the one or more polyurea-containing compounds obtained according to (iii) in R_{C}, wherein the weight ratio of water relative to the one or more polyurea-containing compounds is in the range of from 1:1 to 50:1, preferably in the range of from 5:1 to 20:1, more preferably in the range of from 10:1 to 18:1, obtaining one or more corresponding polyamines.
35. The process of embodiment 33 or 34, wherein the hydrolysis is performed at a temperature in the range of from 150 to 350° C., preferably in the range of from 210 to 290° C., more preferably in the range of from 230 to 270° C.;
   wherein preferably the hydrolysis is performed for a duration in the range of from 0.1 to 15 h, more preferably in the range of from 0.25 to 10 h, more preferably in the range of from 0.5 to 8 h.
36. The process of any one of claims 33 to 35, wherein the hydrolysis is performed at a pressure in the range of from 20 to 70 bar (abs), preferably in the range of from 25 to 65 bar (abs), more preferably in the range of from 30 to 60 bar (abs).
37. The process of any one of embodiments 1 to 32, wherein the cleavage reaction conditions according to (iv) are acidic cleavage conditions.
38. The process of embodiment 37, wherein (iv) comprises admixing a Bronsted acid, wherein the Bronsted acid is preferably selected from the group consisting of hydrochloric acid, a sulfonic acid and a mixture thereof, with the mixture U comprising the one or more polyurea-containing compounds obtained according to (iii) in R_{C}, obtaining one or more corresponding polyisocyanates.
39. The process of any one of embodiments 1 to 38, as far as embodiments 39 is dependent on any one of embodiments 33 to 36, wherein (iv) comprises
   (iv.1) subjecting the mixture U comprising the one or more polyurea-containing compounds obtained according to (iii) to cleavage reaction conditions, preferably hydrolysis reaction conditions, obtaining a mixture comprising one or more corresponding polyamines;
   (iv.2) passing the mixture obtained according to (iv.1) through an evaporation unit EU1, obtaining from EU1 a vapor mixture M_{V1} comprising water and at least a portion of the one or more primary amines, and a liquid mixture M_{EU1} comprising the one or more polyamines;
   (iv.3) passing the vapor mixture M_{V1} obtained according to (iv.2) through a heat exchanger for condensation, obtaining a liquid mixture comprising water and at least a portion of the one or more primary amines, preferably passing said obtained liquid mixture into a tank unit or a stripping column for removal of carbon dioxide;
   (iv.4) passing the liquid mixture obtained according to (iv.3) through PU used according to (v), obtaining a vapor mixture comprising the one or more primary amines depleted in water;
   (iv.5) recycling at least a part of the vapor mixture obtained according to (iv.4) from PU into R_{A} in (i.2) as the first solvent.
40. The process of embodiment 39, wherein the evaporation unit EU1 used in (iv.2) is one or more of a flash tank and a stripping column, preferably a flash tank or a stripping column.
41. The process of embodiment 39 or 40, further comprising
   passing the liquid mixture M_{EU1} comprising the one or more polyamines obtained according to (iv.2) in a heat exchanger, and introducing the obtained mixture in a decanter D, obtaining a lower phase comprising the one or more polyamines and an upper phase comprising water and at least a portion of the one or more primary amines; preferably passing the upper phase through a compressor and subsequently the purification unit PU, more preferably PU2 as defined in embodiment 44.
42. The process of embodiment 41, further comprising
   introducing the lower phase into a purification unit PUA, preferably a distillation column, obtaining a liquid mixture comprising the one or more polyamines, wherein preferably from 99 to 100 weight-%, more preferably from 99.5 to 100 weight-%, more preferably from 99.9 to 100 weight-%, of said mixture consist of the one or more polyamines and obtaining a vapor mixture comprising at least a portion of the one or more primary amines;
   preferably introducing the vapor mixture into PU, more preferably PU2 as defined in embodiment 44.
43. The process of any one of embodiments 1 to 42, wherein, prior to (v), the process further comprises storing M_{V} obtained according to (ii) in a storage tank T3.
44. The process of any one of embodiments 1 to 43, wherein the purification unit PU comprises a first purification sub-unit PU1 and a second purification sub-unit PU2, preferably PU1 and PU2 are serially coupled sub-units.
45. The process of embodiment 44, wherein (v) comprises
   (v.1) introducing M_{V} obtained according to (ii) into PU1, obtaining a vapor mixture A1 comprising the one or more primary amines and a liquid mixture comprising S;
   (v.2) introducing the vapor mixture A1 obtained according to (v.1) into PU2, obtaining a vapor mixture A comprising the one or more primary amines, wherein the concentration of primary amines in A is greater than the concentration of the primary amines in A1.
46. The process of embodiment 45, further comprising
   storing the liquid mixture comprising S obtained according to (v.1) in a storage tank T2 prior to be recycled and use as the solvent in (ii) and/or (iii. 1), preferably in (ii) and (iii. 1).
47. The process of any one of embodiments 44 to 46, wherein (iv) comprises
   (iv.1) subjecting the mixture U comprising the one or more polyurea-containing compounds obtained according to (iii) to cleavage reaction conditions, obtaining a mixture comprising one or more corresponding polyamines;
   (iv.2) passing the mixture obtained according to (iv.1) in an evaporation unit EU1, obtaining from EU1 a vapor mixture M_{V1} comprising water and at least a portion of the one or more primary amines, and a liquid mixture comprising the one or more polyamines;
   (iv.3) passing the vapor mixture M_{V1} obtained according to (iv.2) through a heat exchanger for condensation, obtaining a liquid mixture comprising water and at least a portion of the one or more primary amines, preferably passing said obtained liquid mixture into a tank unit or a stripping column for removal of carbon dioxide;
   (iv.4) passing the liquid mixture obtained according to (iv.3) in PU2 used according to (v), obtaining a vapor mixture comprising the one or more primary amines depleted in water;
   (iv.5) recycling at least a part of the vapor mixture obtained according to (iv.4) from PU into R_{A} in (i.2) as the first solvent.
48. The process of any one of embodiments 1 to 47, further comprising
   passing the mixture P obtained according to (iii) in a purification unit PUP for separating the one or more polyols from the liquid mixture P, obtaining a mixture, comprising the one or more poylols, depleted from at least a portion of compounds other than polyols.
49. The process of embodiment 48, wherein the purification unit PUP comprises, preferably is, a distillation column.
50. The process of embodiment 46 or 47, as far as embodiment 48 or 49 depends on embodiment 24, wherein (iv) comprises
   passing the liquid mixture P obtained according to (ii) in a purification unit PUP, obtaining a liquid mixture, comprising one or more polyols, depleted from at least a portion of the solvent S and a vapor mixture Vs comprising at least a portion S.
51. The process of embodiment 50, wherein (iv) further comprises
   passing the vapor mixture Vs comprising at least a portion of the solvent S and water in a purification unit PU1, obtaining a liquid mixture comprising the solvent;
   introducing the obtained liquid mixture in a storage tank T2, preferably prior to be used as S in (ii.1) and/or (ii.2).
52. The process of any one of embodiments 1 to 51, being a semi-batch process.

In the context of the present invention, a polyurethane can be abbreviated PUR or PU, referring to a class of polymers comprising carbamate (urethane) links. Further, a polyisocyanurate can be abbreviated as PIR, referring to a polymer comprising isocyanurate groups.

In the context of the present invention, ureas are organic compounds with the formula (RR′N)₂CO, wherein R and R′ independently from one another is hydrogen or an organic residue, for example an alkyl residue or an aryl residue. Thus, this definition includes the specific chemical compound ((H₂N)₂CO).

Further, in the context of the present invention, PMDI relates to polymeric diphenylmethane diisocyanate, also known as technical MDI, being a mixture of methylenediphenyl isocyanates and homologous aromatic polyisocyanates. Thus, it is a mixture of compounds with several (typically up to 6) phenylene groups, each of which carrying an isocyanate group.

Furthermore in the context of the present invention, the term “polyurea-containing compound” refers to a compound, preferably a polymer, comprising at least two urea groups. In particular, a polyurea-containing compound, preferably a polyurea-containing polymer, in the context of the present invention is preferably a compound derived from a polyurethane which was subjected to aminolysis.

In the context of the present invention, the term “primary amine” encompasses “primary mono-amine” and “primary poly-amine”. As well-known in the art, primary amine refers to compound having one or more amino groups, wherein the nitrogen atom is directly bond with only one C atom.

In the context of the present invention, an alkyl group consists of carbon atoms and hydrogen atoms.

Further, in the context of the present invention, a term “X is (or comprises) one or more of A, B and C”, wherein X is a given feature and each of A, B and C stands for specific realization of said feature, is to be understood as disclosing that X is (or comprises) either A, or B, or C, or A and B, or A and C, or B and C, or A and B and C. In this regard, it is noted that the skilled person is capable of transfer to above abstract term to a concrete example, e.g. where X is a chemical element and A, B and C are concrete elements such as Li, Na, and K, or X is a temperature and A, B and C are concrete temperatures such as 10° C., 20° C., and 30° C. In this regard, it is further noted that the skilled person is capable of extending the above term to less specific realizations of said feature, e.g. “X is (or comprises) one or more of A and B” disclosing that X is (or comprises) either A, or B, or A and B, or to more specific realizations of said feature, e.g. “X is (or comprises) one or more of A, B, C and D”, disclosing that X is either A, or B, or C, or D, or A and B, or A and C, or A and D, or B and C, or B and D, or C and D, or A and B and C, or A and B and D, or B and C and D, or A and B and C and D.

The present invention is further illustrated by the following examples.

### EXAMPLES

### Reference Example 1: Determination of FTIR Spectra

IR spectroscopy was carried out to determine the conversion of the amination and aminolysis reaction.

For urea synthesis, the absence of the NCO-stretching vibration at 2200-2400 cm⁻¹ indicates complete conversion of a respective isocyanate component (e.g. M20S).

For aminolysis, the area ratio of the urethane vibration (1700-1750 cm⁻¹) before and after the reaction correlates to the conversion.

For solubility tests, the characteristic feature to evaluate the solubility of a polyurea was the presence of the characteristic carbonyl vibration (1600-1700 cm⁻¹) in the sample phase. The area of this vibration correlates to the mass fraction of the urea. For determining the carbonyl vibration as characteristic feature for the solubility of urea, the IR apparatus was calibrated with a 1 weight-% solution of dibutylamine and a n-butylamine.

### Reference Example 2: Determination of ¹³C-NMR Spectra

To evaluate the purity of a synthesized urea ¹³C-NMR spectroscopy was carried out. The absence of peaks in the range of 50 until 80 ppm chemical shift indicates the absence of polyols. The sample was solved in DMSO. As reference standard tetramethylsilane was used.
Reference Example 3: Appliance and Construction Foams and their Compositions a) PUR-appliance foam: A model foam was prepared by mixing 56 weight-% Lupranat® M20S, (Lupranat® M20S is a commercial product of BASF, having a typical NCO content of 31.5 g/100 g and a viscosity of about 200 mPas at 25° C.), and 35 weight-% polyols, a mixture of polyols 1, 2 and 3, containing additionally the additives required for foaming, e.g. catalysts, surfactants, blowing agents.
b) PIR-construction foam: A model foam was prepared by mixing 71.4 weight-% Lupranat® M50S, (Lupranat® M50S is a commercial product of BASF, having a typical NCO content of 31.5 g/100 g and a viscosity of about 550 mPas at 25° C.), and 18.4 weight-% polyols, polyols 4 being the main polyol, including additionally additives required for foaming, e.g. catalysts, surfactants, blowing agents.
c) Polyols (see FIG. 2):
   Polyol 1 is a polyether-polyol synthesized from vicinal-TDA (toluenediamine) as starter and PEO and PPO side chains having a OH-number of 160 mg KOH/g
   Polyol 2 is a polyether-polyol synthesized from vicinal-TDA (toluenediamine) as starter and PPO side chains; having a OH-number of 400 mg KOH/g
   Polyol 3 is a mixture of two polyether-polyols, wherein one polyether-polyol is synthesized from sorbitol as starter and PPO side chains and the other polyether-polyol is synthesized from glycerol as starter and PPO side chains having a OH-number of 430 mg KOH/
   Polyol 4 is a slightly branched polyester polyol based on terephthalic acid and with OH-number of 245 mg KOH/g).
d) Surfactants: silicone oils;
e) Blowing agents: water, cyclopentane;
f) Catalysts: Lupragen® N100, Lupragen® N206, Lupragen® N600 (all commercially available from BASF); These catalysts are usually tertiary amines with the general formular R₁R₂R₃N. The fragments R₁R₂R₃ are alkyl side chains with a chain length of 3-10 carbon atoms.

### Reference Example 4: End-of-Life Foam

a) PUR-appliance end-of-life (EoL) foam:foam powder (MDI-based) obtained from a refrigerator dismantling company
b) PU-flexible conventional foam from a mattress dismantling company TDI-based);
c) Polyols:

| Polyol type | Type | Composition |
|---|---|---|
| Polyether-polyol | Sugar | Sorbitol-PPO |
| Polyether-polyol | TDA | TDA-PPO, TDA-PPO-PEO |
| Polyether-polyol | Amine | Ethylenediamine-PPO |
| Polyether-polyol | Soft | Gly-PPO |
| Polyester-polyol | Ester | Phthalic acid/ |
| | | Diethylenglycol (DEG) |
| Polyether-/ | Hybrid | Biodiesel/Sucrose |
| polyester-polyol | | |
| Polyether-polyol | MDA | MDA-PPO |

### Reference Example 5: Solubility Tests

Four different model systems were prepared for conducting solubility tests of the respective resulting urea in presence of polyether/polyester-polyols.

### 5.1. Synthesis of Model Systems

5.1.a Polyurea Derived from PMDI (Using a Secondary Amine)

In a 2 L round flask 50 g water-free dibutylamine (DBA) and 300 g monochlorobenzene (MCB) were mixed and stirred continuously. Via a dropping funnel a solution consisting of 17 g polymeric isocyanate (Lupranat® M20S having a typical NCO content of 31.5 g/100 g and a viscosity of about 200 mPas at 25° C.; BASF) and 281 g monochlorobenzene was added dropwise over a time period of 1 h. The reaction temperature was increased from 22.7 to 30.1° C. during the reaction. Afterwards, the reaction mixture was stirred overnight. The obtained product was analyzed using FTIR-spectroscopy. The absence of the NCO-stretching vibration in the range of from 2200-2400 cm⁻¹ indicated complete conversion of the isocyanate groups. Monochlorobenzene and excess dibutylamine were evaporated under vacuum and a highly viscous residue was obtained. The yield was 33.9 g corresponding to 98%.

5.1.b Polyurea Derived from PMDI (Using a Primary Amine)

In a 2 L round flask 50 g water-free n-octylamine and 450 g monochlorobenzene were mixed and stirred continuously. Via a dropping funnel a solution consisting of 16 g polymeric isocyanate (Lupranat® M20S having a typical NCO content of 31.5 g/100 g and a viscosity of about 200 mPas at 25° C.; BASF) and 307 g monochlorobenzene was added dropwise over a time period of 1 h. The reaction temperature was increased from 22.7 to 50.8° C. during the reaction. Afterwards the reaction mixture was stirred overnight. The obtained product was analyzed using FTIR-spectroscopy. The absence of the NCO-stretching vibration in the range of from 2200-2400 cm⁻¹ indicated complete conversion of the isocyanate groups. Monochlorobenzene and excess n-octylamine were evaporated under vacuum and a crystalline solid was obtained. The yield was 32.0 g corresponding to 98%.

5.1.c Polyurea Derived from PMDI (Using a Primary Amine)

In a 2 L round flask 41 g water-free n-butylamine and 364 g monochlorobenzene were mixed and stirred continuously. Via a dropping funnel a solution consisting of 22 g polymeric isocyanate (Lupranat® M20S having a typical NCO content of 31.5 g/100 g and a viscosity of about 200 mPas at 25° C.; BASF) and 406 g monochlorobenzene was added dropwise over a time period of 1 h. The reaction temperature was increased from 21.7 to 31.4° C. during the reaction. Afterwards the reaction mixture was stirred overnight. The obtained product was analyzed using FTIR-spectroscopy. The absence the NCO-stretching vibration in the range of from 2200-2400 cm⁻¹ indicated complete conversion of the isocyanate groups. The monochlorobenzene and excess n-butylamine were evaporated under vacuum and a crystalline solid was obtained. The yield was 34.1 g corresponding to 98%.

5.1.d Polyurea Derived from PMDI (Using a Primary Amine)

In a 2 L round flask 84 g water-free aniline and 762 g monochlorobenzene were mixed and stirred continuously. Via a dropping funnel a solution consisting of 36 g polymeric isocyanate (Lupranat® M20S having a typical NCO content of 31.5 g/100 g and a viscosity of about 200 mPas at 25° C.; BASF) and 652 g monochlorobenzene was added dropwise over a time period of 1 h. Afterwards the reaction mixture was stirred overnight. The obtained product was analyzed using FTIR-spectroscopy. The absence of the NCO-stretching vibration in the range of from 2200-2400 cm⁻¹ indicated complete conversion of the isocyanate groups. The monochlorobenzene and excess aniline was evaporated under vacuum and a crystalline solid was obtained. The yield was 61.2 g corresponding to 98%.

5.1.e Polyurea Derived from PMDI (Using a Primary Amine)

In a 2 L round flask 33.1 g water-free toluenediamine (TDA-aromatic polyamine) and 1100 g monochlorobenzene were mixed and stirred continuously. Via a dropping funnel a solution consisting of 3.6 g polymeric isocyanate (Lupranat® M20S having a typical NCO content of 31.5 g/100 g and a viscosity of about 200 mPas at 25° C.; purchased from BASF) and monochlorobenzene (12 g) was added dropwise within 1 h. Afterwards the reaction mixture was stirred overnight. The obtained product was analyzed using FTIR-spectroscopy. The absence of the NCO-stretching vibration at 2200-2400 cm⁻¹ indicated complete conversion of the isocyanate groups. The monochlorobenzene and excess TDA was evaporated under vacuum and a crystalline solid (6.9 g) was obtained. The yield was 99%.

5.1.f Polyurea Derived from PMDI (Using a Primary Amine)

In a 2 L round flask 33 g water-free ethylenediamine (aliphatic polyamine) and 300 g monochlorobenzene were mixed and stirred continuously. Via a dropping funnel a solution consisting of 22 g polymeric isocyanate (Lupranat® M20S having a typical NCO content of 31.5 g/100 g and a viscosity of about 200 mPas at 25° C.; purchased from BASF) and monochlorobenzene (340 g) was added dropwise within 1 h. Afterwards the reaction mixture was stirred overnight. The obtained product was analyzed using FTIR-spectroscopy. The absence of the NCO-stretching vibration at 2200-2400 cm⁻¹ indicated complete conversion of the isocyanate groups. The monochlorobenzene and excess ethylenediamine was evaporated under vacuum and a crystalline solid (29.8 g) was obtained. The yield was 93%.

### 5.2. Evaluation of Polyurea Solubility

### 5.2.a Solubility Screening

Approximately 5 g of a polyurea according to a respective model system, 2.5 g polyether-polyol-mixture, containing TDA, glycerol and sucrose derived polyether-polyols and 25 g of a respective solvent were mixed at room temperature. After 90 min stirring the polyether-polyols were added and an IR sample was taken, diluted with chloroform, and analyzed. The area of the carbonyl vibration in the range of from 1600-1700 cm⁻¹ correlates with the solved amount of urea. The detection limit was <1.0 weight-%. The results for the solubility tests for the model systems prepared according to 1.a-d are shown in table 1 below.

TABLE 1
Results for the solubility tests for the model systems prepared according to 5.1.a-f.

| | Polyurea acc. to | Polyurea acc. to | Polyurea acc. to | Polyurea acc. to | | |
|---|---|---|---|---|---|---|
| | Ref. Ex. 5.1.a | Ref. Ex. 5.1.c | Ref. Ex. 5.1.b | Ref. Ex. 5.1.d | | |
| | (secondary amine) | (primary amine) | (primary amine) | (primary amine) | Polyurea acc. to | Polyurea acc. to |
| Solvent | [wt.-%] | [wt.-%] | [wt.-%] | [wt.-%] | Ref. Ex. 5.1.e | Ref. Ex. 5.1.f |
| Methyl-tert.- | 8.8 | <1.0 | <1.0 | <1.0 | <1.0 | <1.0 |
| butylether (MTBE) | | | | | | |
| Methanol | — | <1.0 | <1.0 | <1.0 | <1.0 | <1.0 |
| Ethanol | 10.1 | — | — | — | — | — |
| Cyclohexane | 2.2 | <1.0 | <1.0 | <1.0 | <1.0 | <1.0 |
| Water | 12.3 | <1.0 | <1.0 | <1.0 | <1.0 | <1.0 |
| Benzene | — | <1.0 | <1.0 | <1.0 | <1.0 | <1.0 |
| Toluene | 9.1 | — | — | — | — | — |
| Xylene | — | <1.0 | <1.0 | <1.0 | <1.0 | <1.0 |

The results of the solubility tests (table 1), show that the polyurea derived from the reaction of a secondary amine, in particular dibutylamine, with PMDI was soluble in the tested solvents whereas the polyureas derived from the reaction of a primary amine with PMDI were practically not soluble. In particular, it was found that 9.1 weight-% of the polyurea derived from the reaction of dibutylamine with PMDI could be dissolved in toluene. It can thus be expected that comparable solubility would be achieved in xylene. In contrast thereto, less than 1.0 weight-% of the polyurea derived from the reaction of a primary amine, in particular n-octylamine, n-butylamine and aniline as used in Reference Examples 1.b-1.d, were soluble in xylene. It is thus considered that the polyreas of these examples are not soluble in the tested solvents.

### Example 1: Aminolysis of PUR-Appliance Foam with n-Butylamine

30 g of the PUR-appliance foam according to Reference Example 3 a) was milled using a coffee grinder and dried overnight at 100° C. in a nitrogen flushed atmosphere under atmospheric pressure. Afterwards the dried foam and 150 g n-butylamine were charged into a stirred autoclave. The aminolysis reaction was conducted at 140° C. and approximately 5 bar (depending on the vapor pressure of n-butylamine) for 75 min. After the reaction the mixture was cooled down to room temperature and approximately 120 mL of n-butylamine were evaporated. To the resulting residue 150 g xylene were added and the precipitation of the polyurea started. To complete the precipitation, the remaining n-butylamine was evaporated. Subsequently the precipitated polyurea was filtered off and washed several times with xylene. With the aid of ¹³C-NMR- and IR-spectroscopy the conversion and the purity of the precipitate were documented. As can be seen from the IR spectra shown in FIG. 2 no characteristic urethane vibration was detected for the polyurea. The conversion was about 95% and only small amounts of ether groups could be detected, as can be seen from the ¹³C-NMR spectrum shown in FIG. 3. The purity was higher than 90%. 16.1 g of the corresponding polyurea-containing compound was obtained (yield: 61%).

### Example 2: Aminolysis of PUR-Appliance Foam with Aniline

10 g of the PUR-appliance foam according to Reference Example 3 a) was milled using a coffee grinder and dried overnight at 100° C. in a nitrogen flushed atmosphere under atmospheric pressure. Afterwards the dried foam and 110 g aniline were charged into a 0.5 L round flask. The aminolysis reaction was conducted at 180° C. under reflux for 1 h. During the aminolysis reaction all compounds were completely dissolved in aniline. After the reaction the mixture was cooled down to room temperature and the formed polyurea started precipitating. After 12 h the precipitate was filtered off. The solids were washed several times with xylene and dried overnight, yielding 3.5 g polyurea corresponding to 40%. Excess aniline was evaporated from the filtrate, and 250 g xylene was added to the residue. The solution was stirred overnight. The newly formed precipitate was filtrated, washed and dried, yielding 2.1 g polyurea corresponding to 24%. With the aid of ¹³C-NMR- and IR-spectroscopy the aminolysis conversion and the purity of the precipitate were documented. The conversion was about 98% and no ether groups could be detected. The purity was higher than 95%. 5.6 g of the corresponding polyurea-containing compound was obtained (yield: 64%).

### Example 3: Aminolysis of PUR-Appliance End-of-Life (EoL) Foam with n-Butylamine

24 g of the PUR-appliance end-of-life (EoL) foam according to Reference Example 4 a) and 120 g n-butylamine were charged into a stirred autoclave. The aminolysis reaction was conducted at 140° C. and approximately 5 bar (depending on the vapor pressure of n-butylamine) for 75 min. After the reaction the mixture was cooled down to room temperature. After pressure relaxation the mixture was heated to 60° C. and filtered over a nylon filter to remove insoluble inorganic impurities and organic impurities (mᵢₘₚᵤᵣᵢₜᵢₑₛ=2.0 g). Then, approximately 120 mL of n-butylamine were evaporated. To the resulting residue 150 g xylene were added and after some time the precipitation of the polyurea started. To complete the precipitation, the remaining n-butylamine was evaporated. Subsequently the precipitated polyurea was filtered off and washed several times with xylene. The aminolysis conversion and the purity of the precipitate were analyzed using ¹³C-NMR- and IR-spectroscopy. The conversion was about 98% and no ether groups could be detected. The purity was higher than 95%. 8.6 g of the corresponding polyurea-containing compound was obtained.

### Example 4: Aminolysis of PUR-Appliance End-of-Life (EoL) Foam with Aniline

25 g of the PUR-appliance end-of-life (EoL) foam according to Reference Example 4 a) and 250 g aniline were added to a 0.5 L round flask. The aminolysis reaction was conducted at 180° C. under reflux for 1 h. During the aminolysis reaction inorganic impurities precipitated. After the reaction the mixture was filtrated using a nylon filter and the filtrate was cooled down to room temperature (mᵢₘₚᵤᵣᵢₜᵢₑₛ=2.1 g). Then, the formed polyurea started to precipitate. After 12 h the precipitate was filtered off. The solids were washed several times with xylene and dried overnight (m_{polyurea,1}=10.3 g). Excess aniline was evaporated from the filtrate, and 250 g xylene were added to the residue. The solution was stirred overnight. The resulting precipitate was filtered off, washed, and dried. (m_{polyurea,2}=1.8 g, Yield=24%). Using ¹³C-NMR- and IR-spectroscopy the conversion and purity of the precipitate were analyzed. The conversion was about 90% and no ether groups could be detected. The purity was higher than 95%, 12.1 g of the corresponding polyurea-containing compound was obtained.

### Example 5: Aminolysis of PU Flexible Foam with n-Butylamine

15 g of the PU flexible foam according to Reference Example 4 b) was mixed with liquid nitrogen, then milled using a coffee grinder and dried overnight at 100° C. in a nitrogen flushed atmosphere under atmospheric pressure. Afterwards the dried foam and 150 g of n-butylamine were added to a stirred autoclave. The aminolysis reaction was conducted at 140° C. and approximately 5 bar (depending on the vapor pressure of n-butylamine) for 75 min. After the reaction the mixture was filtered at 140° C. under pressure to remove solid impurities. Then, n-butylamine was evaporated under vacuum and the remaining residue washed with n-heptane several times. Using ¹³C-NMR- and IR-spectroscopy the conversion and purity of the precipitated polyurea were analyzed. The conversion was about 90% and no ether groups could be detected. The purity was higher than 80%. 6.1 g of the corresponding polyurea-containing compound was obtained.

### Example 6: Aminolysis of a Polyisocyanurate (PIR) Construction Foam with Aniline

15 g of the dried and shredded PIR-construction foam according to Reference Example 3 b) a polyisocyanurate construction foam and 400 g aniline were charged into a round flask equipped with a reflux condenser. The aminolysis reaction was conducted at 180° C. for 60 min until the foam components were completely dissolved in aniline. The mixture was cooled down to room temperature and the polyurea started precipitating. The precipitated polyurea was filtered, and the filtrate was concentrated by evaporative removal of aniline. The remaining solids were also filtered off and washed several times with acetone. Using ¹³C-NMR- and IR-spectroscopy the conversion and purity of the precipitate were analyzed. The conversion was about 90% and no ether groups could be detected. The purity was higher than 95%, 12.2 g of the corresponding polyurea-containing compound was obtained (yield: 60%).

### Example 7: Hydrolysis of Polyurea-Containing Compound (Polymer) with Water

5.1 g of polyurea obtained from Example 3 and 75 g water were charged into a pressure autoclave. The mixture was heated to 250° C. and the temperature was hold for 5 h, whereby the pressure was approximately 40 bar(abs). After cooling down the mixture was analyzed using GC to evaluate the amount of PMDA. The purity was determined based on the area ratios of the GC peaks, accordingly the purity was 85% (m_{residue}=4.3 g).

### DESCRIPTION OF THE FIGURES

FIG. 1 is a schematic representation of a production unit used for the process according to preferred embodiments of the invention.

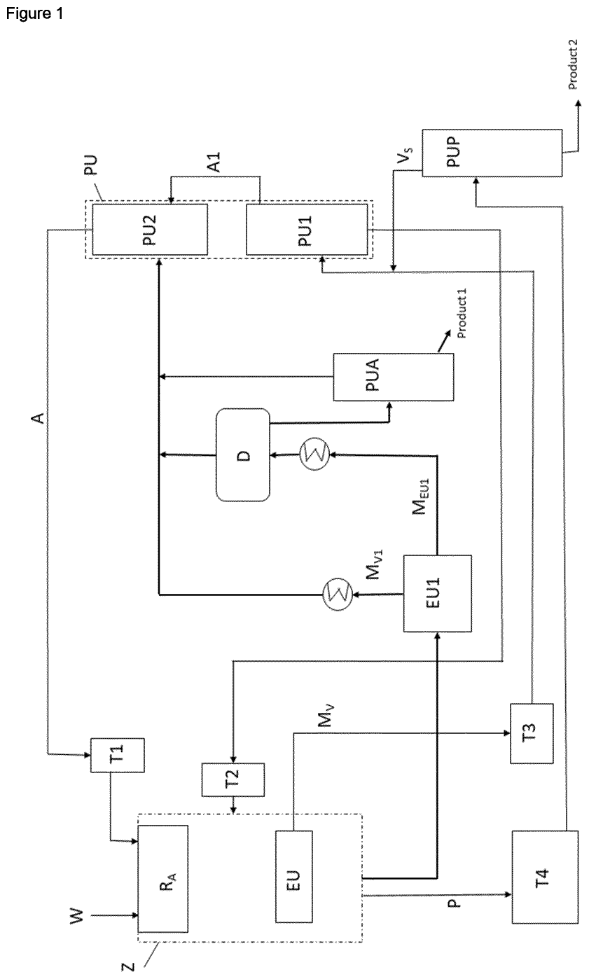

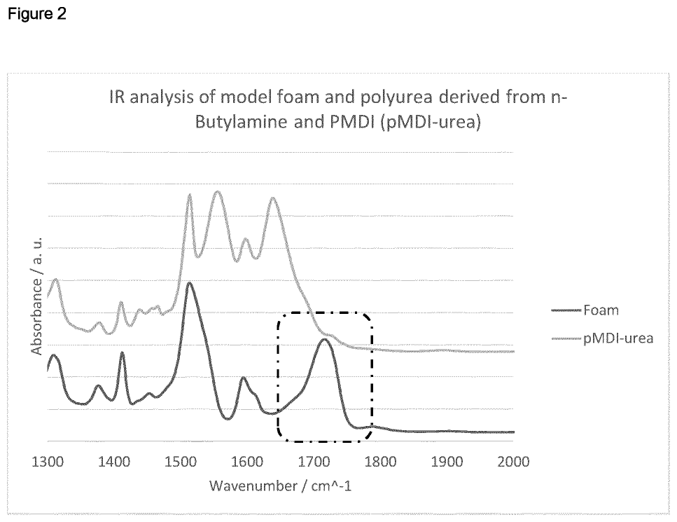

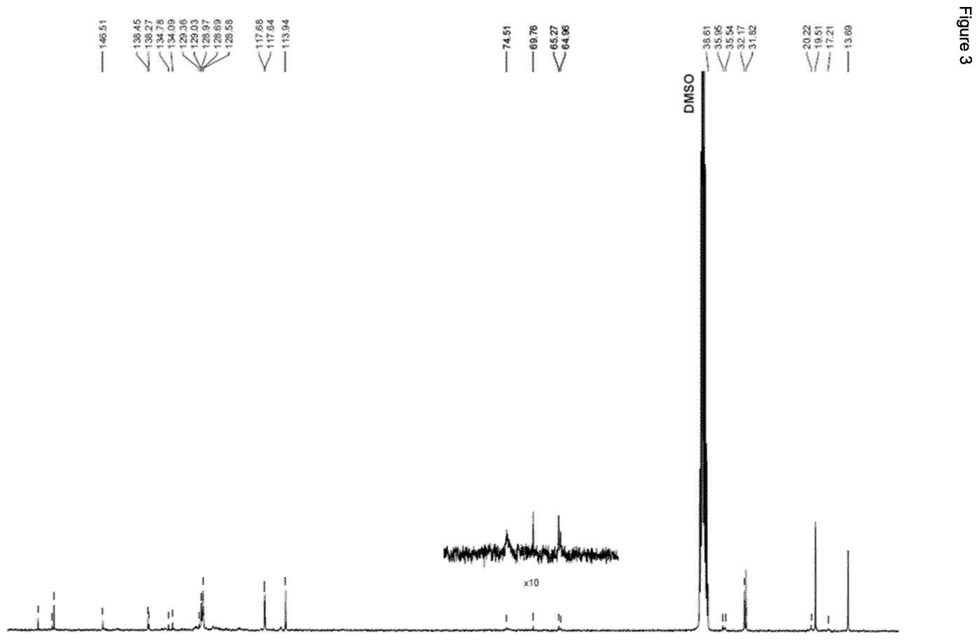

The production unit comprises a zone Z, wherein the aminolysis and the cleavage reactions are performed batch wise, said zone comprises among other a reaction unit R_{A} and an evaporation unit EU. The other components of this zone are defined in the present invention in the foregoing and are not represented in the figure. Further, the production unit comprises four different storage tanks T1, T2, T3 and T4. T1 is a storage tank for the one or more primary amines, preferably aniline or n-butylamine, T2 is a storage tank for the solvent S, preferably xylene, T3 is a storage tank for the vapor mixture M_{V}, comprising solvent and primary amines and T4 is a storage tank for the polyols formed in Z, said tank comprises a mixture of the polyols and S. At the beginning of the process, the solid material W, preferably waste material (foam), are introduced into R_{A} with one or more primary amines from T1, this permits to obtain a mixture M comprising one or more polyols as well as one or more polyurea-containing compounds. Further treatments are performed such as the addition of a solvent S, preferably xylene, to the mixture M for subsequent flashing in EU. The vapor mixture M_{V} is removed from EU and stored in T3. The mixture comprising one or more polyols as well as one or more polyurea-containing compounds is further precipitated by the addition of S and the polyols and the polyurea-containing compounds are separated. This steps are not shown in FIG. 1. Further, the polyurea-containing compounds are subjected to cleavage reaction conditions, in this particular preferred embodiment to hydrolysis, to obtain the corresponding polyamines. The polyamines mixture is removed from Z and introduced in an evaporation EU1, obtaining M_{V1} and M_{EU1}. M_{V1} and M_{EU1} are as defined in the foregoing. Preferably M_{V1} comprises aniline or n-butylamine and M_{EU1} comprises the polyamines and one or more of water and aniline or n-butylamine. M_{V1} is then passed through a heat exchanger for condensation and in a purification sub-unit PU2. The mixture A comprising, preferably consisting of, the primary amines (aniline or n-butylamine) is removed from PU2 and introduced into T1. Thus, the primary amines are recycled. Further, M_{EU1} is passed through a heat exchanger and a decanter D, obtaining an upper phase comprising water and the one or more polyamines and a lower phase comprising the polyamines. The lower phase is then introduced into a purification unit PUA for obtaining the final polyamines (Product 1). The upper phase is optionally passed through a compressor and then into PU2 for recovering the primary amines as mixture A. As to the recycling of S, preferably xylene, M_{V} after having been stored in T3 is passed through a purification unit PU1 and the primary amine rich fraction A1 is further passed through PU2 for recovering the primary amines as mixture A. The solvent, preferably xylene, is removed at the bottom of PU1 and recycled into T2 for further use in the claimed process. Finally, the mixture P comprising the polyols is stored in a storage tank T4 prior to be passed through a purification unit PUP for obtaining the final polyols (Product 2) and a mixture Vs comprising the solvent S, preferably xylene, which is further passed through PU1.

FIG. 2 shows the spectra of IR analysis of the model foam and the polyurea derived from PMDI (pMDI-urea) according to Example 1. On the abscissa, the wavenumber is shown in cm⁻¹ and on the ordinate the absorbance is given in arbitrary units.

FIG. 3 shows the ¹³C-NMR spectrum of the polyurea derived from PMDI according to Example 1. On the abscissa, the chemical shift is given in ppm and on the ordinate the absorbance is given in arbitrary units.

### CITED LITERATURE

WO 2008/014988 A1
Kunststoffhandbuch [Plastics handbook], volume 7, Polyurethane [Polyurethanes]”, Carl Hanser Verlag, 3^{rd} edition 1993, chapter 3.4.4 and 3.4.6 to 3.4.1
WO 2006/034800
EP 0090444
WO 2005/090440

1.-18. (canceled)

19. A solvent-efficient process for recycling one or more polymers, selected from the group consisting of polyurethanes, polyurethane ureas, polyisocyanurates, and a mixture of two or more thereof, the one or more polymers being contained in a solid material W, the process comprising:
(i) preparing a mixture M comprising one or more polyurea-containing compounds, and further comprising one or more polyols, comprising:
(i.1) providing a solid material W comprising one or more polymers selected from the group consisting of polyurethanes, polyurethane ureas, polyisocyanurates, and a mixture of two or more thereof;
(i.2) introducing the solid material W provided in (i.1) and one or more primary amines, as a first solvent, in a reactor unit R_{A}, obtaining a mixture;
(i.3) subjecting the mixture obtained according to (i.2) in R_{A} to aminolysis reaction conditions, obtaining a mixture M comprising one or more polyurea-containing compounds, and further comprising one or more polyols and one or more primary amines; and
(i.4) removing the mixture M obtained according to (i.3) from R_{A};
(ii) passing a second solvent S and the mixture M obtained according to (i) into an evaporation unit EU, obtaining, from the evaporation unit EU, a vapor mixture M_{V} comprising S and at least a portion of the one or more primary amines, and a liquid mixture M_{L} comprising the one or more polyurea-containing compounds, and the one or more polyols;
(iii) isolating the one or more polyurea-containing compounds from M_{L} obtained according to (ii), obtaining a mixture U comprising the one or more polyurea-containing compounds and a mixture P comprising the one or more polyols;
(iv) subjecting the mixture U comprising the one or more polyurea-containing compounds obtained according to (iii) to cleavage reaction conditions in a reaction unit R_{C}, obtaining either one or more corresponding polyamines or one or more corresponding polyisocyanates;
(v) separating the one or more primary amines from M_{V} obtained according to (ii) in a purification unit PU, obtaining a vapor mixture A comprising the one or more primary amines depleted in S; and
(vi) recycling at least a part of A obtained according to (v) from PU into R_{A} in (i.2) as the first solvent.

20. The process of claim 19, wherein the one or more primary amines used in (i.2), as the first solvent, are selected from the group consisting of aliphatic monoamines, aliphatic polyamines, aromatic monoamines, and mixtures of two or more thereof.

21. The process of claim 20, wherein the one or more primary amines are aliphatic monoamines having the formula H₂NR¹,
wherein R¹ is selected from the group consisting of (C₃-C₂₅)alkyl, phenyl, (C₇-C₂₅) aralkyl, and (C₇-C₂₅)alkaryl; or
wherein the one or more primary amines are aromatic monoamines, wherein the aromatic monoamines are selected from the group consisting of aniline, toluidine, naphtylamine, and mixtures of two or more thereof.

22. The process of claim 19, wherein the atoms forming the one or more primary amines used in (i.2) are C, H and N; and
wherein the second solvent S used in (ii) has a water content in the range of from 0 to 1000 ppm.

23. The process of claim 19, wherein the ratio of the weight of the solid material W introduced into R_{A} relative to the weight of the one or more primary amines introduced into R_{A} is in the range of from 1:100 to 1:1.

24. The process of claim 19, wherein at most 0.1 weight-% of the mixture M obtained according to (i) consist of polymer selected from the group consisting of polyurethane, polyurethane urea and polyisocyanurate.

25. The process of claim 19, further comprising, after (i.4) and prior to (ii), passing the mixture M removed from R_{A} according to (i.4) into a solid-liquid separation unit, obtaining a liquid mixture M_{SLS} comprising the one or more polyurea-containing compounds and the one or more polyols and a solid mixture comprising impurities;
wherein passing the mixture M removed from R_{A} according to (i.4) into a solid-liquid separation unit is performed at a temperature in the range of from 50 to 250° C.;
wherein (ii) comprises:
passing a second solvent S and the liquid mixture M_{SLS} into an evaporation unit EU, obtaining, from the evaporation unit EU, a vapor mixture M_{V} comprising S and at least a portion of the one or more primary amines, and a liquid mixture M_{L} comprising the one or more polyurea-containing compounds, and the one or more polyols.

26. The process of claim 19, wherein S used in (ii) is selected from the group consisting of hydrocarbons, ketones and ether; and/or wherein S has a water content in the range of from 0 to 1000 ppm.

27. The process of claim 19, wherein (iii) comprises:
(iii.1) adding a solvent to the liquid mixture M_{L} obtained according to (ii), allowing the one or more polyurea-containing compounds to precipitate, obtaining a mixture comprising one or more precipitated polyurea-containing compounds; and
(iii.2) passing the mixture comprising the one or more precipitated polyurea-containing compounds obtained according to (iii.1) through a solid-liquid separation unit SLU, obtaining a liquid mixture P comprising the one or more polyols, and a solid mixture U comprising the one or more polyurea-containing compounds,
wherein at most 0.1 weight-% of the mixture U consist of polyol; and
wherein, in the process the atoms forming the one or more primary amines used in (i.2) are C, H and N and the second solvent S used in (ii) has a water content in the range of from 0 to 1000 ppm.

28. The process of claim 19, wherein the cleavage reaction conditions according to (iv) are hydrolysis reaction conditions.

29. The process of claim 28, wherein (iv) comprises
admixing water with the mixture U comprising the one or more polyurea-containing compounds obtained according to (iii) in R_{C}, wherein the weight ratio of water relative to the one or more polyurea-containing compounds is in the range of from 1:1 to 50:1, obtaining one or more corresponding polyamines.

30. The process of claim 19, wherein (iv) comprises:
(iv.1) subjecting the mixture U comprising the one or more polyurea-containing compounds obtained according to (iii) to cleavage reaction conditions, obtaining a mixture comprising one or more corresponding polyamines;
(iv.2) passing the mixture obtained according to (iv.1) in an evaporation unit EU1, obtaining from EU1 a vapor mixture M_{V1} comprising water and at least a portion of the one or more primary amines, and a liquid mixture M_{EU1} comprising the one or more polyamines;
(iv.3) passing the vapor mixture M_{V1} obtained according to (iv.2) through a heat exchanger for condensation, obtaining a liquid mixture comprising water and at least a portion of the one or more primary amines;
(iv.4) passing the liquid mixture obtained according to (iv.3) in PU used according to (v), obtaining a vapor mixture comprising the one or more primary amines depleted in water; and
(iv.5) recycling at least a part of the vapor mixture obtained according to (iv.4) from PU into R_{A} in (i.2) as the first solvent.

31. The process of claim 30, further comprises:
passing the liquid mixture M_{EU1} comprising the one or more polyamines obtained according to (iv.2) in a heat exchanger, and introducing the obtained mixture in a decanter D, obtaining a lower phase comprising the one or more polyamines and an upper phase comprising water and at least a portion of the one or more primary amines.

32. The process of claim 31, further comprising:
introducing the lower phase into a purification unit PUA, obtaining a liquid mixture comprising the one or more polyamines, and obtaining a vapor mixture comprising at least a portion of the one or more primary amines.

33. The process of claim 19, wherein the purification unit PU comprises a first purification sub-unit PU1 and a second purification sub-unit PU2.

34. The process of claim 33, wherein (v) comprises:
(v.1) introducing M_{V} obtained according to (ii) into PU1, obtaining a vapor mixture A1 comprising the one or more primary amines and a liquid mixture comprising S; and
(v.2) introducing the vapor mixture A1 obtained according to (v.1) into PU2, obtaining a vapor mixture A comprising the one or more primary amines.

35. The process of claim 33, wherein (iv) comprises:
(iv.1) subjecting the mixture U comprising the one or more polyurea-containing compounds obtained according to (iii) to cleavage reaction conditions, obtaining a mixture comprising one or more corresponding polyamines;
(iv.2) passing the mixture obtained according to (iv.1) in an evaporation unit EU1, obtaining from EU1 a vapor mixture M_{V1} comprising water and at least a portion of the one or more primary amines, and a liquid mixture comprising the one or more polyamines;
(iv.3) passing the vapor mixture M_{V1} obtained according to (iv.2) through a heat exchanger for condensation, obtaining a liquid mixture comprising water and at least a portion of the one or more primary amines;
(iv.4) passing the liquid mixture obtained according to (iv.3) in PU2 used according to (v), obtaining a vapor mixture A comprising the one or more primary amines depleted in water; and
(iv.5) recycling at least a part of the vapor mixture A obtained according to (iv.4) from PU into R_{A} in (i.2) as the first solvent.

36. The process of claim 34, wherein (iv) comprises:
passing the liquid mixture P obtained according to (ii) in a purification unit PUP, obtaining a liquid mixture, comprising one or more polyols, depleted from at least a portion of the solvent S and a vapor mixture Vs comprising at least a portion S;
wherein (iv) further comprises:
passing the vapor mixture Vs comprising at least a portion of the solvent S and water in a purification unit PU1, obtaining a liquid mixture comprising the solvent;
introducing the obtained liquid mixture in a storage tank T2.
